(19) United States
(12) Patent Application Publication
(10) Pub. No.: US 2009/0012300 A1
(43) Pub. Date: Jan. 8, 2009

(54) Process for the Preparation of an (Hetero) Arylamine

(76) Inventors: Mathilda Maria Henrica Lambers, Weert (NL); Ben De Lange, Munstergeleen (NL); Adreas Hendrikus Maria Vries, Maastricht (NL); Johannes Gerardus Vries, Maastricht (NL); Natascha Sereinig, Eindhoven (NL)

Correspondence Address: NIXON & VANDERHYE, PC, 901 NORTH GLEBE ROAD, 11TH FLOOR, ARLINGTON, VA, 22203, US

(21) Appl. No.: 11/632,121

(22) PCT Filed: Jul. 15, 2005
(86) PCT No.: PCT/NL05/00512

§ 371 Date: Feb. 20, 2008

(30) Foreign Application Priority Data
Jul. 16, 2004 (EP) 04077068.7

(51) Int. Cl. C07D 211/12; C07D 233/58

(52) U.S. Cl. 546/192; 548/343.5

(57) ABSTRACT

The present invention relates to a process for the preparation of an (hetero)arylamine, wherein an optionally substituted (hetero)aromatic bromide compound is contacted with a nucleophilic organic nitrogen-containing compound in the presence of a base, and a catalyst comprising a copper atom or ion and at least one ligand, said ligand comprising at least one coordinating oxygen atom, and if said oxygen atom is part of an OH group, then said OH group is attached to an aliphatic spa carbon atom or to a vinylic carbon atom.

The present invention relates to a process for the preparation of an (hetero)aryl amine according to formula (3) wherein an optionally substituted (hetero)aromatic bromide compound according to formula (1) is contacted with a nucleophilic organic nitrogen-containing compound according to formula (2) in the presence of a base, and a catalyst comprising a copper atom or ion and at least one ligand.

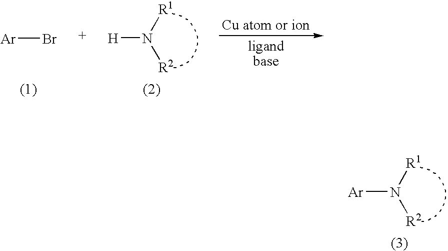

Ar in formulae (1) and (3) stands for an optionally substituted aromatic or heteroaromatic group. R¹ and R² are as defined below. The “dotted line” in the structures of formulae (2) and (3) stands for an optional connection between R¹ and R².

(Hetero)aryl amines according to formula (3) are important substructures in agrochemical and pharmaceutical products.

Kwong et al., Organic Letters 2002, Vol. 4, No. 4, 581-584 discloses a copper-catalyzed amination reaction of aryl iodides when using cuprous iodide as the catalyst and ethylene glycol as the ligand. However, it is disclosed that the copper-catalyzed amination reaction of aryl iodides is not successful when propylene and butylene glycols are used as ligands. Kwong et al., Organic Letters 2002, Vol. 4, No. 4, 581-584 further discloses the copper-catalyzed amination of arylbromides, in which phenolic ligands proved more efficient ligands than ethylene glycol. Arylbromides could be used if the reaction was conducted using a large excess of the amine as the solvent.

Kwong and Buchwald, Organic Letters 2003, Vol. 5, No. 6, 793-796 discloses a copper-catalyzed amination of aryl bromides by using cuprous iodide as the catalyst and diethylsalicylamide as an example of a phenolic ligand. However, said reaction proved to work well when primary amines are employed as substrates, but not when secondary amines are used.

A disadvantage of the known copper-catalyzed amination reactions of aryl iodides is that aryl iodides are expensive and generate relatively large waste amounts. Moreover, the use of amine-containing ligands may hinder the work up process, in particular the separation of the amine-containing ligand from the amine end product tends to be difficult.

The disadvantages of the known copper-catalyzed amination reactions of arylbromides are that phenolic ligands are toxic and large excess of the amine may need to be used.

Buchwald et al. in US 2003/0065187 A1 disclose that copper-catalyzed aminations of arylbromides without use of large excess of the amines or toxic phenolic ligands, require ligands which contain at least one nitrogen atom, as shown in FIGS. 13, 14, 15, 16 and 26 in US 2003/0065187. However, disadvantages of the nitrogen containing ligands are that the ligands may be arylated by the arylbromide and therefore lower yields of the amine end product are obtained. The arylated ligands are amine-containing products, whereby separation of this unwanted side product from the amine end product tends to be difficult.

It is an object of the invention to provide an inexpensive, simple and commercially attractive process for the preparation of an (hetero)aryl amine according to formula (3).

This has been achieved according to the process of the present invention by using a ligand that comprises at least one coordinating oxygen atom, and if said oxygen atom is part of an OH group, then said OH group is attached to an aliphatic sp³ carbon atom or to a vinylic carbon atom. The ligand according to the invention does not comprise a nitrogen atom.

With the term “coordinating atom” is meant that the atom is capable of electronic and/or spatial interaction with a copper atom or ion, preferably by donating electron density to a copper atom or ion.

Surprisingly, it has been found that with the aid of this process, copper-catalyzed amination reactions of relatively inexpensive arylbromides according to formula (1) can be achieved under mild conditions with commercially attractive ligands and with acceptable yields. This is particularly surprising because such bromide compounds are known to be much less reactive than the corresponding much more expensive iodide compounds. Such favourable results are obtained that a relatively inexpensive process can be developed that in practice is easy to scale up and therefore is pre-eminently suitable for commercial applications.

It has further been surprisingly found that with the aid of the present process, a high amount or concentration of compound (1) can be converted with relatively high yield into the desired end product (3). This is highly advantageously when to be applied for industrial scale production.

In the process of the present invention, the ligand comprises at least one coordinating oxygen atom, and if said oxygen atom is part of an OH group, then said OH group is attached to an aliphatic SP3 carbon atom or to a vinylic carbon atom and the ligand does not comprise a nitrogen atom. The oxygen atom, when not part of an OH group, is preferably connected to a carbon atom.

Preferably, the ligand is at least a bidentate ligand comprising at least two coordinating atoms wherein the oxygen atom is the first coordinating atom and wherein the second coordinating atom is selected from the group consisting of oxygen, phosphorus, and sulphur. Preferably, the at least bidentate ligand is e.g. a chelating ligand comprising at least two coordinating atoms with a spatial relationship there between, such that the coordinating atoms are capable of interacting simultaneously with a copper atom or ion. A further advantage of the at least bidentate ligand in the process of the present invention is that a more stable electronic and/or spatial interaction may take place with a copper atom or ion. More preferably, the ligand is at least a bidentate ligand comprising at least two coordinating oxygen atoms. The ligand may also serve as a solvent in the process of the present invention.

Suitable monodentate ligands in the process of the invention are ethers, ketones or sp³-C alcohols, for example di-isopropylether, methylisobutylketone, tertiair-butyl methyl ether, tertiar-butanol, mixtures thereof, or the like.

Suitable bidentate ligands in the process of the present invention are α-diketones, β-diketones, γ-diketones, α-ketoesters, β-ketoesters, α-ketoamides, β-ketoamides, α-di-esters, β-di-esters, hydroxyketones, hydroxy ethers or alkoxy alcohols, diols, hydroxythioethers, mixtures thereof, and the like. Examples of suitable β-diketones are 2,4-pentanedione, 2,2,6,6-tetramethyl-3,5-heptanedione, 1,3-cyclohexanedione, 2-methyl-1,3-cyclohexanedione, and the like. A preferred β-diketone in the process of the present invention is 2,4-pentanedione. Examples of suitable α-diketones are 2,3-butanedione, 1,2-cyclohexanedione, and the like. Examples of suitable β-ketoesters are tertiair-butyl-acetoacetate, methyl-acetoacetate, and the like. Examples of suitable β-di-esters are di-tertiair-butyl malonate, di-ethyl malonate, and the like. Examples of suitable diols are, for example, glycol, ethylene glycol, 1,2- and 1,3-propanediol; 1,2-, 1,3- and 1,4-butanediol and 1,2-hexanediol; substituted diols, such as for example pinacol and cis- and trans-1,2-cyclohexanediol. A preferred diol in the present process is ethylene glycol. Examples of suitable hydroxythioethers are ethyl 2-hydroxyethyl sulfide, amyl 2-hydroxyethylsulfide, 2-hydroxyethyl sulfate and the like. Further examples of suitable bidentate ligands according to the invention are 2-[1,3,2]dioxaphospholane-2-yl-ethanol, 3-[1,3,2]phosphaoxinane-2-yl-propanol or 2-hydroxyethyl phosphate.

Examples of suitable tridentate ligands are triols, such as, for example, glycerol, 1,4,7-trioxonane, mixtures thereof, and the like.

Examples of suitable tetra- and polydentate ligands are, for example, glucose, sucrose, fructose and crown-ethers, such as, for example, 1,4,7,10-tetraoxacyclododecane, 1,4,7,10,13-pentaoxacyclopentadecane or 1,4,7,10,13,16-hexaoxacyclooctadecane, mixtures thereof, and the like.

In the process of the present invention, a combination of two or more ligands as disclosed above may be used together with a copper catalyst. Also, a combination of one or more of the ligands of the invention with any other ligand, such as, for example, phosphorus-containing ligands, for example, phosphines, e.g. triphenylphosphine; phosphites, e.g. triethylphosphite, tri-isopropylphosphite; phosphonites, e.g. phenyl-O,O-di-o-tolylphosphonite, 2,10-dimethoxy-4,8-dimethyl-6-phenyl-5,7-dioxa-6-phospha-dibenzo[a, c]cycloheptene; phosphinites, e.g. diphenyl, O-cyclohexylphosphinite and phosphoramidites, e.g. 1-benzo[1,3,2]dioxaphosphol-2-yl-pyrrolidine, and the like, may be used. Other examples of such additional ligands are dienes, such as norbornadiene or CO.

The catalyst used in the process of the present invention comprises a copper atom or ion and at least one ligand as defined above.

Examples of catalysts comprising a copper atom or ion that can be used in the process of the present invention are copper metal or organic or inorganic compounds of copper(I) or copper(II). Suitable examples of copper catalysts in the process of the invention are copper(I)chloride, copper(II) chloride, copper(I)bromide, copper(II) bromide, copper(I)iodide, copper(II) iodide, basic copper(II)carbonate, copper(I)nitrate, copper(II)nitrate, copper(II)sulphate, copper(I)sulfide, copper(II)sulfide, copper(I)acetate, copper(II)acetate, copper(I)oxide, copper(II)oxide, copper(I)trifluoroacetate, copper(II)trifluoroacetate, copper(I)benzoate, copper(II)benzoate, and copper(II)trifluoromethyl sulphonate. Preferred are copper(I)chloride, copper(II)chloride, copper(I)bromide and copper(II)bromide. These catalysts are readily available and relatively inexpensive.

The copper atom or ion and the ligand of the catalyst may be added to the reaction mixture separately or simultaneously, or they may be added in the form of a preformed catalyst complex. A suitable example of a preformed catalyst complex is Cu(II)(2,4-pentanedione)₂.

The molar ratio between the copper salt and the optionally substituted (hetero)aromatic bromide compound (1) lies between 0.00001 and 30 mol %, preferably between 0.01 and 15 mol %, more preferably between 0.1 and 10 mol %, and most preferably between 1 and 5 mol %.

The ratio between the ligand and the copper atom may suitably be 0.1 or higher, preferably, between 1 and 10 and more preferred between 1 and 3.

The process of the present invention involves an optionally substituted (hetero)aromatic bromide compound according to formula (1). The (hetero)aromatic group Ar may suitably contain at least 1 carbon atom in its cycle, preferably at least 2 carbon atoms, more preferably at least 3, even more preferred at least 4 carbon atoms in its cycle. The (hetero)aromatic group may be mono- or polycyclic, and may be a carbocycle or a heterocycle containing at least one of the heteroatoms P, O, N or S. Suitable examples of (hetero)aromatic groups from which the bromide compound has been derived are phenyl, naphthyl, pyridyl, pyrrolyl, quinolyl, isoquinolyl, furyl, thienyl, benzofuryl, indenyl, pyrimidinyl, pyrazolyl and imidazolyl. The (hetero)aromatic group can optionally be substituted with one or more substituents, in principle all substituents which are inert under the given reaction conditions. Suitable examples of such substituents are an alkyl group with for example 1 to 20 carbon atoms, for example a methyl, ethyl, isobutyl or trifluoromethyl group; an alkenyl group with for example 2 to 20 carbon atoms; a (hetero)aryl group with for example 1 to 50 carbon atoms; a carboxyl group; an alkyl or aryl carboxylate group with for example 2 to 50 carbon atoms; a formyl group; an alkanoyl or aroyl group with for example 2 to 50 carbon atoms; a carbamoyl group; an N-substituted alkyl or aryl carbamoyl group with for example 2 to 50 carbon atoms; an amino group; an N-substituted alkyl or arylamino group with for example 1 to 50 carbon atoms; a formamido group; an alkyl or aryl amido group with for example 2 to 50 carbon atoms; a hydroxy group; an alkoxy or aryloxy group with for example 1 to 50 carbon atoms; cyano; nitro; halogen and an alkyl or arylthio group with for example 1 to 50 carbon atoms.

Suitable examples of optionally substituted (hetero)aromatic bromide compounds of formula (1) are, for example, bromobenzene, bromopyridines, for example 3-bromopyridine; bromobenzonitriles, for example 2-bromobenzonitrile or 4-bromobenzonitrile; bromonitrobenzenes, for example 4-bromonitrobenzene; 2-bromo-6-methoxynaphthalene and bromoanisoles, for example 4-bromoanisole, 4-bromo-biphenyl, 5-bromo-m-xylene, and the like, or any mixtures thereof.

The process of the present invention further involves a nucleophilic organic nitrogen-containing compound according to formula (2) as substrate, which compound may be chosen from

(i) primary amines,
(ii) secondary amines,
(iii) hydrazine derivatives, or any combination thereof. Mixtures of two or more of compounds (i), (ii) and (iii) may be used as well.

### (i) Primary or (ii) Secondary Amines

The primary or secondary amines can be represented by the general formula (2):

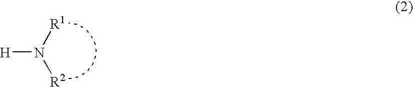

wherein at most one of R¹ or R² represents a hydrogen atom, and wherein independently from each other, R¹ and R² may represent an optionally substituted hydrocarbon group containing 1 to 20 carbon atoms, which may be linear or branched, saturated or unsaturated acyclic aliphatic group, a monocyclic or polycyclic, saturated, unsaturated or aromatic carbocyclic or heterocyclic group; or a concatenation of said groups; or wherein R¹ and R² can be bonded to constitute, with the carbon atoms carrying them, a carbocyclic or heterocyclic group containing 3 to 20 monocyclic or polycyclic, saturated or unsaturated atoms.

In case of a saturated heterocyclic compound (2), the compound may contain one or more heteroatoms such as nitrogen, oxygen, sulphur or phosphorus, at least one of which is a nucleophilic NH, such as, for example, piperazines, morpholines, oxazolidines, e.g. 2-oxazolidone, imidazolidines and the like.

The secondary amine may also be a heteroaromatic compound. The heteroaromatic compound may be mono- or polycyclic, wherein at least one of the carbon atoms is replaced by at least one atom chosen from the list consisting of a nitrogen, oxygen, sulphur or phosphorus atom. The heteroaromatic compound may be substituted or not. The monocyclic heteroaromatic compound may in particular contain 5 or 6 atoms in the cycle and possibly contain 1, 2 or 3 heteroatoms such as nitrogen, oxygen, sulphur or phosphorus, at least one of which is a nucleophilic NH. The polycyclic heteroaromatic compound is constituted by at least one aromatic cycle and contains at least one heteroatom in at least one cycle (aromatic or non aromatic cycle), at least one of which is a nucleophilic NH.

Suitable amines may be amines of formula HN—R¹R² in which R¹, R², which may be identical or different, represent a C¹ to C¹⁵ alkyl group, preferably C¹ to C¹⁰ alkyl, more preferably C¹ to C⁴ alkyl, a C³ to C⁸ cycloalkyl group or a C⁶ to C¹² aryl or arylalkyl group, such as for example phenyl, naphthyl or benzyl groups. Specific examples are benzylamine, aniline, N-methylaniline, diphenylamine, dibenzylamine and butylamine. Further suitable amines are saturated heterocyclic secondary amines such as, for example, pyrrolidine, piperidine, morpholine, piperazine, N-methylpiperazine, N-acetyl piperazine, and the like. Further suitable amines are heteroaromatic secondary amines such as, for example, imidazole, benzimidazole, pyrazole, triazole e.g. 1,2,4-1H-triazole, tetrazole e.g. 1-H-tetrazole, and the like.

(iii) Hydrazine Derivatives

The nucleophilic nitrogen-containing compound according to formula (2) may also be a hydrazine derivative, wherein R¹ is hydrogen and R² may be presented by any one of the groups (2a), (2b) or (2c):

—NH—COOR³ (2a)

—NH—COR⁴ (2b)

—N═CR⁵R⁶ (2c)

in which R³ to R⁶ may be identical or different, and may have the meanings of R¹ and R² as defined for the primary and secondary amines under paragraphs (i) and (ii) above. Preferably, R³ to R⁸ represent a C¹ to C¹⁵ alkyl group, preferably a C¹ to C¹⁰ alkyl, more preferably a C³ to C⁸ cycloalkyl group or a C⁶ to C¹² aryl or arylalkyl group, Preferably, R³ represents a tertiair-butyl group or a benzyl group, R⁴ represents a methyl or phenyl group and R⁵, R⁶ represent a phenyl group.

The number of moles of the nucleophilic nitrogen-containing compound (2) to the number of moles of the (hetero)aromatic bromide compound (1) is usually in the range of 0.6 to 5, preferably, 0.9 to 2.0, more preferably 1.0-1.5.

The process of the present invention is carried out in the presence of a base. Examples of suitable bases are, for example, mentioned in Modern Synthetic Methods for Copper-Mediated C(aryl)-O, C(aryl)-N, C(aryl)-S Bond Formation, Ley, S. V.; Thomas A. W. Angew. Chem. Int. Ed. 2003, 42, 5400-5449 or in “Handbook of Chemistry and Physics, 66^{th} Edition, p. D-161 and D-162”. In general, any Bronsted base may be used in the process of the present invention. The pkA of the base is preferably 2 or higher, more preferably between 3 and 50, and even more preferred between 5 and 30. The base is preferably chosen from bases and basic salts from alkali metals and earth alkali metals, more preferably from the group of (earth)alkali metal carbonates, and (earth)alkali metal hydrogen carbonates, (earth)alkali metal acetates, (earth)alkali metal hydroxides, (earth)alkali metal alkoxides, and (earth)alkali metal phosphates. Surprisingly, in the presence of bases and basic salts from alkali metals and earth alkali metals, a relatively high weight % of the (hetero)aromatic bromide compound (1) can be converted with relatively high conversion and yield into the desired product (3). Moreover, the reaction will occur relatively faster. This is highly advantageously when to be applied for large industrial scale production. The base is preferably selected from bases and basic salts from alkali metals and earth alkali metals Na, K, Ca and Mg. More preferred, the base is chosen from K₂CO₃, NaOAc, KOAc, Na₂CO_{3.} CaCO₃, K₃PO₄, NaHCO₃, Li₂CO₃, and Cs₂CO₃. Especially preferred bases are K₂CO₃, Na₂CO_{3.} K₃PO₄, NaOAc and KOAc, since these bases are readily available and inexpensive and result in relatively high yields, especially at a high concentration of substrate compound (1). Most preferred bases are K₂CO₃, Na₂CO₃ and K₃PO₄

Suitable solvents that can be used in the process according to the invention are solvents that do not react under the reaction conditions, for example polar solvents, such as for example ethers, amides and the like, or hydrocarbons, such as toluene. Also a mixture of solvents may be used. Particularly suitable solvents are aprotic polar solvents, for example, N-methyl pyrrolidinone (NMP), dimethyl formamide (DMF), dimethyl acetamide (DMA), dimethyl sulphoxide (DMSO), acetonitrile, glymes, for example ethyleneglycol dimethylether, and the like. N-methyl-pyrrolidinone (NMP) is a particularly suitable solvent in the process of the present invention. Furthermore, NMP is an environmental friendly solvent. In specific cases reactants, ligands and/or products can serve as a solvent.

According to one preferred embodiment of the present invention, the present process works surprisingly well (relatively high yield and relatively fast reaction) if the weight % of the (hetero)aromatic bromide compound (1) is at least 10% relative to the total weight of the components of the reaction mixture. Preferably, the weight % of compound (1) relative to total weight of the components of the reaction mixture is at least 15%, more preferred at least 17%, even more preferred at least 20%, and most preferred at least 30%.

Preferably, the amounts of moles of the (hetero)aromatic bromide compound (1) per litre of solvent is in the range of 0.8-10 mole, more preferred from 1.5-7 mole, and most preferred between 3 and 6 mole.

The process according to the invention may be applied in the presence of one or more additives like, surfactants, such as phase-transfer catalysts, such as, for example quaternary ammonium salts, in particular tetrabutylammonium chloride or bromide, triethylbenzylammonium bromide, or tetraethylammonium chloride, salts, and the like. Other possible additives are salts, such as for example lithiumchloride. The process according to the invention may be applied by using external stimuli, for example by microwave heating, ultrasound or light.

The temperature at which the process according to the invention is carried out is not particularly critical. One skilled in the art can determine the optimum temperature for the specific reaction system. Preferably the reaction temperature lies between 15 and 250° C., more preferably between 25 and 175° C., most preferably between 50 and 125° C.

The process of the present invention is generally carried out at atmospheric pressure or in a closed vessel. The process is preferably carried out in a nitrogen atmosphere.

The order in which the reagents are added is not critical. One suitable order may be that in which the catalyst, the ligand, the nucleophilic nitrogen-containing compound (2), the base, the (hetero)aromatic bromide compound (1) and optionally the solvent are charged. Then, the reaction mixture is heated to the desired temperature. Another suitable order may be by charging the catalyst, the base, the (hetero)aromatic bromide compound (1) and optionally the solvent and adding the nucleophilic nitrogen-containing compound (2) thereto.

The product obtained with the process of the present invention may be further purified by methods commonly known in the art, for example, by extraction, crystallization, distillation or chromatography

The separation of the catalyst from the reaction mixture may, for example, be accomplished by extraction, filtration, decanting or centrifuging.

With the process of the present invention, the (hetero)arylamine compound (3) may be obtained with relatively high conversion and yield.

The yield obtained with the process of the present invention is preferably at least 30%, more preferred at least 40%, even more preferred at least 50%, particularly preferred at least 60% and most preferred at least 80%.

Compound (3) may be used as an intermediate in agrochemical and pharmaceutical products, in electronic devices, and the like.

The invention will be elucidated on the basis of the examples, without however being limited by them.

### DEFINITIONS

C_{end}=number of moles of product (3) formed at the end of the reaction.
D₀=number of moles of optionally substituted (hetero)aromatic bromide compound (1) at the start of the reaction.
Dₑ=number of moles of optionally substituted (hetero)aromatic bromide compound (1) at the end of the reaction.

The yield (%) may be defined by formula (4):

Yield (%)=C_{end}/D₀*100 (4)

The conversion (%) may be defined by formula (5):

Conversion (%)=(D₀−Dₑ)/D₀*100 (5)

The selectivity may be defined by formula (6):

Selectivity (%)=(yield/conversion)*100 (6)

### EXAMPLE IA

### N-(phenyl)benzylamine: Arylation of Bromobenzene with Benzylamine, 2,4-Pentanedione as Ligand and K₂CO₃ as Base (Concentration 4.80 Mol Bromobenzene/L NMP)

A 50 mL reactor was charged successively with 10.05 g (72.7 mmol) K₂CO_{3.} 780 mg CuCl (7.9 mmol), 11.2 g (71.2 mmol) bromobenzene, 15 mL NMP and 1.78 g (18.0) mmol 2,4-pentanedione. The reactor was flushed with nitrogen and then kept under a slow stream of nitrogen. Then 10.28 g (9.61 mmol) benzylamine was added. The reaction mixture was heated until 110° C. and kept at this temperature for about 18 h. Samples were taken regularly and analyzed by GC using bromobenzene and N-(phenyl)benzylamine as external standard. GC analysis after 18 h: Conversion based on bromobenzene 90%, yield N-(phenyl)benzylamine 90%.

### Comparative Experiment 1A

### N-(phenyl)benzylamine: Arylation of Bromobenzene with Benzylamine, Diacetamide as Ligand and K₂CO₃ as Base (Concentration 4.80 Mol Bromobenzene/L NMP)

A 50 mL reactor was charged successively with 10.05 g (72.7 mmol) K₂CO_{3.} 780 mg CuCl (7.9 mmol), 11.2 g (71.2 mmol) bromobenzene, 15 mL NMP and 1.82 g (18.0) mmol diacetamide. The reactor was flushed with nitrogen and then kept under a slow stream of nitrogen. Then 10.28 g (9.61 mmol) benzylamine was added. The reaction mixture was heated until 110° C. and kept at this temperature for about 70 h. Samples were taken regularly and analyzed by GC using bromobenzene and N-(phenyl)benzylamine as external standard. GC analysis after 18 h: Conversion based on bromobenzene 95%, yield N-(phenyl)benzylamine 68% (after 70 h the yield was 74%).

### EXAMPLE IB

### N-(phenyl)benzylamine: Arylation of Bromobenzene with Benzylamine, 2,4-Pentanedione as Ligand and K₂CO₃ as Base (Concentration 0.95 Mol Bromobenzene/L NMP)

A 50 mL reactor was charged successively with 3.69 g (26.7 mmol) K₂CO_{3.}, 0.29 g CuCl (2.9 mmol), 4.10 g (26.1 mmol) bromobenzene, 27.5 mL NMP and 0.65 g (6.5 mmol) 2,4-pentanedione. The reactor was flushed with nitrogen and then kept under a slow stream of nitrogen. Then 3.77 g (35.2 mmol) benzylamine was added. The reaction mixture was heated until 110° C. and kept at this temperature for 18 h. Samples were taken regularly and analyzed by GC using bromobenzene and N-(phenyl)benzylamine as external standard. GC analysis after 18 h: Conversion based on bromobenzene 56%, yield N-(phenyl)benzylamine 43%.

### EXAMPLE IIA

### N-(phenyl)imidazole: Arylation of Bromobenzene with Imidazole, 2,4-Pentanedione as Ligand and K₂CO₃ as Base (Concentration 4.80 Mol Bromobenzene/L NMP)

A 50 mL reactor was charged successively with 10.05 g (72.7 mmol) K₂CO_{3.}, 780 mg CuCl (7.9 mmol), 11.2 g (71.2 mmol) bromobenzene, 15 mL NMP and 1.78 g (18.0) mmol 2,4-pentanedione. The reactor was flushed with nitrogen and then kept under a slow stream of nitrogen. Then 6.33 g (9.3 mmol) imidazole was added. The reaction mixture was heated until 110° C. and kept at this temperature for 20 h. Samples were taken regularly and analyzed by GC using bromobenzene and N-(phenyl)imidazole as external standard. GC analysis after 20 h: Conversion based on bromobenzene 99%, yield N-(phenyl)imidazole 98%.

### EXAMPLE IIB

### N-(phenyl)imidazole: Arylation of Bromobenzene with Imidazole, 2,4-Pentanedione as Ligand and K₂CO₃ as Base (Concentration 0.95 Mol Bromobenzene/L NMP)

A 50 mL reactor was charged successively with 3.69 g (26.7 mmol) K₂CO_{3.}, 0.29 g CuCl (2.9 mmol), 4.10 g (26.1 mmol) bromobenzene, 27.5 mL NMP and 0.65 g (6.5 mmol) 2,4-pentanedione. The reactor was flushed with nitrogen and then kept under a slow stream of nitrogen. Then 2.31 g (33.9 mmol) imidazole was added. The reaction mixture was heated until 110° C. and kept at this temperature for 20 h. Samples were taken regularly and analyzed by GC using bromobenzene and N-(phenyl)imidazole as external standard. GC analysis after 20 h: Conversion based on bromobenzene 90%, yield N-(phenyl)imidazole 89%.

### EXAMPLE IIIA

### N-(phenyl)piperidine: Arylation of Bromobenzene with Piperidine, 2,4-Pentanedione as Ligand and K₂CO₃ as Base (Concentration 4.80 Mol Bromobenzene/L NMP)

A 50 mL reactor was charged successively with 10.05 g (72.7 mmol) K₂CO_{3.}, 780 mg CuCl (7.9 mmol), 11.2 g (71.2 mmol) bromobenzene, 15 mL NMP and 1.78 g (18.0) mmol 2,4-pentanedione. The reactor was flushed with nitrogen and then kept under a slow stream of nitrogen. Then 7.9 g (9.3 mmol) piperidine was added. The reaction mixture was heated until 110° C. and kept at this temperature for 44 h. Samples were taken regularly and analyzed by GC using bromobenzene and N-(phenyl)piperidine as external standard. GC analysis after 44 h: Conversion based on bromobenzene 70%, yield N-(phenyl)piperidine 39%.

### EXAMPLE IIIB

### N-(phenyl)piperidine: Arylation of Bromobenzene with Piperidine, 2,4-Pentanedione as Ligand and K₂CO₃ as Base (Concentration 0.95 Mol Bromobenzene/L NMP)

A 50 mL reactor was charged successively with 3.69 g (26.7 mmol) K₂CO_{3.}, 0.29 g CuCl (2.9 mmol), 4.10 g (26.1 mmol) bromobenzene, 27.5 mL NMP and 0.65 g (6.5 mmol) 2,4-pentanedione. The reactor was flushed with nitrogen and then kept under a slow stream of nitrogen. Then 2.9 g (34.1 mmol) piperidine was added. The reaction mixture was heated until 110° C. and kept at this temperature for 40 h. Samples were taken regularly and analyzed by GC using bromobenzene and N-(phenyl)piperidine as external standard. GC analysis after 40 h: Conversion based on bromobenzene 94%, yield N-(phenyl)piperidine 17%.

### Result:

Surprisingly, using a higher concentration of compounds (1) and (2) in the process of the present invention (Ex. IA versus IB, IIA versus IIB and IIIA versus IIIB) results in a higher yield of compound (3).

### EXAMPLE IV

### N-(phenyl)imidazole: Arylation of Bromobenzene with Imidazole with Glycol as Ligand (Concentration 5.0 Mol Bromobenzene/L NMP)

A 5 mL flask was charged successively with 760 mg (5.5 mmol) K₂CO_{3.}, 50 mg CuCl (0.5 mmol), 785 mg (5.0 mmol) bromobenzene, 1 mL NMP and 620 mg (10 mmol) glycol. The reactor was flushed with nitrogen and then kept under a slow stream of nitrogen. Then 442 mg (6.5 mmol) imidazole was added. The reaction mixture was heated until 125° C. and kept at this temperature for 16 h. GC analysis using dihexylether as internal standard indicated: Conversion based on bromobenzene 90%, yield N-(phenyl)imidazole 90%.

### EXAMPLE V

### N-(phenyl)benzylamine: Arylation of Bromobenzene with Benzylamine, Glycol as Ligand and K₂CO₃ as Base (Concentration 5.0 Mol Bromobenzene/L NMP)

A 5 mL flask was charged successively with 760 mg (5.5 mmol) K₂CO_{3.}, 50 mg CuCl (0.5 mmol), 785 mg (5.0 mmol) bromobenzene, 1 mL NMP and 620 mg (10 mmol) glycol. The reactor was flushed with nitrogen and then kept under a slow stream of nitrogen. Then 696 mg (6.5 mmol) benzylamine was added. The reaction mixture was heated until 125° C. and kept at this temperature for 16 h. GC analysis using dihexylether as internal standard indicated: Conversion based on bromobenzene 61%, yield N-(phenyl)benzylamine 43%.

### Result:

By comparing the results of Ex. IIA (ligand 2,4-pentanedione) with Ex. IV (ligand glycol) (and similarly Ex. IA with Ex. V), it turns out that both ligands according to the invention result in favourable yields for the process of the present invention.

### EXAMPLE VI

### N-(phenyl)benzylamine: Arylation of Bromobenzene with Benzylamine, T-Butylacetoacetate as Ligand and K₂CO₃ as Base (Concentration 5.0 Mol Bromobenzene/L NMP)

A 5 mL flask was charged successively with 760 mg (5.5 mmol) K₂CO_{3.}, 50 mg CuCl (0.5 mmol), 785 mg (5.0 mmol) bromobenzene, 1 mL NMP and 198 mg (1.25 mmol) t-butylacetoacetate. The reactor was flushed with nitrogen and then kept under a slow stream of nitrogen. Then 696 mg (6.5 mmol) benzylamine was added. The reaction mixture was heated until 120° C. and kept at this temperature for 16 h. GC analysis using dihexylether as internal standard indicated: Conversion based on bromobenzene 42%, yield N-(phenyl)benzylamine 41%.

### EXAMPLE VII

### N-(phenyl)benzylamine: Arylation of Bromobenzene with Benzylamine, Di-T-Butyl-Malonate as Ligand and K₂CO₃ as Base (Concentration 5.0 Mol Bromobenzene/L NMP)

A 5 mL flask was charged successively with 760 mg (5.5 mmol) K₂CO_{3.}, 50 mg CuCl (0.5 mmol), 785 mg (5.0 mmol) bromobenzene, 1 mL NMP and 270 mg (1.25 mmol) di-t-butylmalonate. The reactor was flushed with nitrogen and then kept under a slow stream of nitrogen. Then 696 mg (6.5 mmol) benzylamine was added. The reaction mixture was heated until 123° C. and kept at this temperature for 90 h. GC analysis using dihexylether as internal standard indicated: Conversion based on bromobenzene 83%, yield N-(phenyl)benzylamine 67%.

### EXAMPLE VIII

### N-(phenyl)benzylamine: Arylation of Bromobenzene with Benzylamine, 2-Methyl-1,3-Cyclohexanedione as Ligand and K₂CO₃ as Base (Concentration 5.0 Mol Bromobenzene/L NMP)

A 5 mL flask was charged successively with 760 mg (5.5 mmol) K₂CO_{3.}, 50 mg CuCl (0.5 mmol), 785 mg (5.0 mmol) bromobenzene, 1 mL NMP and 158 mg (1.25 mmol) 2-methyl-1,3-cyclohexanedione. The reactor was flushed with nitrogen and then kept under a slow stream of nitrogen. Then 696 mg (6.5 mmol) benzylamine was added. The reaction mixture was heated until 120° C. and kept at this temperature for 20 h. GC analysis using dihexylether as internal standard indicated: Conversion based on bromobenzene 78%, yield N-(phenyl)benzylamine 58%.

### EXAMPLE IX

### N-(4-methoxyphenyl)benzylamine: Arylation of 4-Bromoanisole with Benzylamine, 2,4-Pentanedione as Ligand and K₂CO₃ as Base (Concentration 2.5 Mol 4-Bromoanisole/L NMP)

A 5 mL flask was charged successively with 760 mg (5.5 mmol) K₂CO_{3.}, 50 mg CuCl (0.5 mmol), 935 mg (5.0 mmol) 4-bromoanisole, 2 mL NMP and 125 mg (1.25 mmol) 2,4-pentanedione. The reactor was flushed with nitrogen and then kept under a slow stream of nitrogen. Then 696 mg (6.5 mmol) benzylamine was added. The reaction mixture was heated until 115° C. and kept at this temperature for 16 h. GC analysis using dihexylether as internal standard indicated: Conversion based on 4-bromoanisole 46%, yield N-(4-methoxyphenyl)benzyl amine 43%.

### EXAMPLE X

According to the procedure described in example IX, 4-bromobenzonitrile was converted in N-(4-cyanophenyl)benzylamine. Conversion based on 4-bromobenzonitril 97%, yield N-(4-cyanophenyl)benzyl amine 60%.

### EXAMPLE XI

According to the procedure described in example IX, 3-bromopyridine was converted in N-(3-pyridine)benzylamine. Conversion based on 3-bromopyridine 48%, yield N-(3-pyridine)benzyl amine 47%

### Result:

The results of Ex. IX, X and XI show that the process of the present invention gives favourable yields for varying compounds (1).

### EXAMPLE XII

### N-(4-methoxyphenyl)imidazole: Arylation of 4-Bromoanisole with Imidazole, 2,4-Opentanedione as Ligand and K₂CO₃ as Base (Concentration 2.5 Mol 4-Bromoanisole/L NMP)

A 5 mL flask was charged successively with 760 mg (5.5 mmol) K₂CO_{3.}, 50 mg CuCl (0.5 mmol), 935 mg (5.0 mmol) 4-bromoanisole, 2 mL NMP and 125 mg (1.25 mmol) 2,4-pentanedione. The reactor was flushed with nitrogen and then kept under a slow stream of nitrogen. Then 443 mg (6.5 mmol) benzylamine was added. The reaction mixture was heated until 115° C. and kept at this temperature for 16 h. GC analysis using dihexylether as internal standard indicated: Conversion based on 4-bromoanisole 73%, yield N-(4-methoxyphenyl)imidazole 52%.

### EXAMPLE XIII

According to the procedure described in example XII, 4-bromobenzonitrile was converted in N-(4-cyanophenyl)imidazole Conversion based on 4-bromobenzonitril 100%, yield N-(4-cyanophenyl)imidazole 53%.

### EXAMPLE XIV

### N-(phenyl)benzylamine: Arylation of Bromobenzene with Benzylamine and 2,2,6,6-Tetramethyl-3,5-Heptanedione as Ligand (Concentration 1 Mol Bromobenzene/L NMP)

A 10 mL flask was charged successively with 1.6 g (5 mmol) Cs₂CO₃, 50 mg CuCl (0.5 mmol), 785 mg (5.0 mmol) bromobenzene, 5 mL NMP and 230 mg (1.25 mmol) 2,2,6,6-tetramethyl 3,5-heptanedione. The reactor was flushed with nitrogen and then kept under a slow stream of nitrogen. Then 750 mg (7 mmol) benzylamine was added. The reaction mixture was heated until 120° C. and kept at this temperature for 10 h. GC analysis using dihexylether as internal standard indicated: Conversion based on bromobenzene 81%, yield N-(phenyl)benzyl amine 80%.

### Result:

Ex. XIV shows an additional variation in ligand which results in favourable yields.

### EXAMPLE XV

According to the procedure described in example XIV, 4-bromobenzonitril was converted in N-(4-cyanophenyl)benzylamine. Conversion based on 4-bromobenzonitril 100%, yield N-(4-cyanophenyl)benzyl-amine 76%.

### EXAMPLE XVI

According to the procedure described in example XIV, 4-bromobiphenyl was converted in N-(4-biphenyl)benzylamine. Conversion based on 4-bromobiphenyl 87%, yield N-(4-biphenyl)benzylamine 78%.

### EXAMPLE XVII

### N-(phenyl)benzylamine: Arylation of Bromobenzene with Benzylamine, 2,4-Pentadione as Ligand, Cs₂CO₃ as Base (Concentration 4.80 Mol Bromobenzene/L NMP)

A 50 mL reactor was charged successively with 23.7 g (72.7 mmol) Cs₂CO_{3.}, 780 mg CuCl (7.9 mmol), 11.2 g (71.2 mmol) bromobenzene, 15 mL NMP and 1.78 g (18.0) mmol 2,4-pentanedione. The reactor was flushed with nitrogen and then kept under a slow stream of nitrogen. Then 10.28 g (9.61 mmol) benzylamine was added. The reaction mixture was heated until 110° C. and kept at this temperature for about 18 h. Samples were taken regularly and analyzed by GC using bromobenzene and N-(phenyl)benzylamine as external standard. GC analysis after 18 h: Conversion based on bromobenzene 80%, yield N-(phenyl)benzylamine 46%.

### Result:

By comparing the results of Ex. IA (high concentration compound (1) and same reagentia, base K₂CO₃) with Ex. XVII (base Cs₂CO₃), it turns out that the use of K₂CO₃ results in a favourable yield at high concentration of substrate compound (1).

### EXAMPLE XVIII

### N-(phenyl)imidazole: Arylation of Bromobenzene with Imidazole, Cu(II)[2,4-pentanedione]₂ as Ligand and K₂CO₃ as Base (Concentration 4.80 Mol Bromobenzene/L NMP)

A 50 mL reactor was charged successively with 10.05 g (72.7 mmol) K₂CO_{3.}, 943 mg (3.6 mmol) Cu(II)-[2,4-pentanedione]₂, 11.2 g (71.2 mmol) bromobenzene and 15 mL NMP. The reactor was flushed with nitrogen and then kept under a slow stream of nitrogen. Then 6.33 g (9.3 mmol) imidazole was added. The reaction mixture was heated until 110° C. and kept at this temperature for 12 h. Samples were taken regularly and analyzed by GC using bromobenzene and N-(phenyl)imidazole as external standard. GC analysis after 12 h: Conversion based on bromobenzene 87%, yield N-(phenyl)imidazole 86%.

1. Process for the preparation of an (hetero)aryl amine according to formula (3) wherein an optionally substituted (hetero)aromatic bromide compound according to formula (1) is contacted with a nucleophilic organic nitrogen-containing compound according to formula (2) in the presence of a base, and a catalyst comprising a copper atom or ion and at least one ligand, 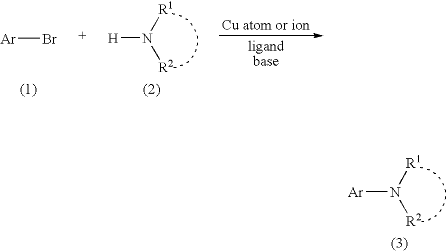
wherein, the ligand comprises at least one coordinating oxygen atom, and if said oxygen atom is part of an OH group, then said OH group is attached to an aliphatic sp³ carbon atom or to a vinylic carbon atom and wherein the ligand does not comprise a nitrogen atom.

2. Process according to claim 1, wherein the ligand is at least a bidentate ligand comprising at least two coordinating atoms wherein the oxygen atom is the first coordinating atom and wherein the second coordinating atom is selected from the group consisting of oxygen, phosphorus, and sulphur.

3. Process according to claim 1, wherein the ligand is at least a bidentate ligand comprising at least two coordinating oxygen atoms.

4. Process according to claim 1, wherein the ligand is a β-diketone selected from the list consisting of 2,4-pentanedione, 2,2,6,6-tetramethyl-3,5-heptanedione, 1,3-cyclohexanedione, 2-methyl-1,3-cyclohexanedione, or any mixture thereof.

5. Process according to claim 1, wherein the nucleophilic organic nitrogen-containing compound (2) is selected from the group consisting of
(i) primary amines or (ii) secondary amines represented by formula (2): 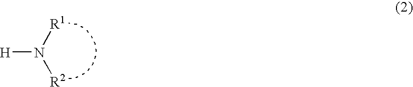
wherein at most one of R¹ or R² represents a hydrogen atom, and wherein independently from each other, R¹ and R² may represent a hydrocarbon group containing 1 to 20 carbon atoms, which may be linear or branched, saturated or unsaturated acyclic aliphatic group, a monocyclic or polycyclic, saturated, unsaturated or aromatic carbocyclic or heterocyclic group; or a concatenation of said groups; or wherein R₁ and R₂ can be bonded to constitute, with the carbon atoms carrying them, a carbocyclic or heterocyclic group containing 3 to 20 monocyclic or polycyclic, saturated or unsaturated atoms, or
(iii) hydrazine derivatives according to formula (2), wherein R¹ is hydrogen and R² may be presented by any one of the groups (2a), (2b) or (2c):
—NH—COOR³ (2a)
NH—COR⁴ (2b)
—N═CR⁵R⁶ (2c)
in which R³ to R⁶ may be identical or different, and may have the meanings of R¹ and R² as defined for the primary amines (i) and secondary amines (ii).

6. Process according to claim 1, wherein the weight % of the (hetero)aromatic bromide compound (1) is at least 10% relative to the total weight of the components of the reaction mixture.

7. Process according to claim 1, wherein the base is chosen from bases and basic salts from alkali metals and earth alkali metals.

8. Process according to claim 7, wherein the base is selected from inorganic bases or basic salts from alkali metals and earth alkali metals Na, K, Ca and Mg.

9. Process according to claim 8, wherein the base is selected from the group consisting of K₂CO_{3.}, Na₂CO₃, K₃PO₄, NaOAc, KOAc or mixtures thereof.

10. Process according to claim 1, wherein the process is carried out in the presence of a solvent that does not react under the reaction conditions.

11. Process according to claim 1, wherein the nucleophilic organic nitrogen-containing compound (2) is selected from the group consisting of benzylamine, imidazole, benzimidazole, 1,2,4-1H-triazole, pyrazole, 1-H-tetrazole, pyrrolidine, morpholine, piperidine, piperazine, N-methylpiperazine, N-acetylpiperazine, 2-oxazolidone or mixtures thereof.
